(19) United States
(12) Patent Application Publication
(10) Pub. No.: US 2016/0024565 A1
(43) Pub. Date: Jan. 28, 2016

(54) FRAGMENT COMPLEMENTATION OF BASED ASSAYS

(71) Applicant: TUFTS UNIVERSITY, Medford, MA (US)

(72) Inventors: Elena Benito-Pena, Madrid (ES); David R. Walt, Boston, MA (US)

(21) Appl. No.: 14/775,418

(22) PCT Filed: Mar. 13, 2014
(86) PCT No.: PCT/US14/26592

§ 371 Date: Sep. 11, 2015

Related U.S. Application Data
(60) Provisional application No. 61/780,713, filed on Mar. 13, 2013.

(51) Int. Cl. C12Q 1/68

(52) U.S. Cl. CPC C12Q 1/6834

(57) ABSTRACT

The present disclosure provides, among other things, methods and compositions for detecting and/or quantifying analytes using fragment complementation technologies. In accordance with various embodiments of the present disclosure, a kit can include a) a capture probe immobilized on a surface, wherein the capture probe can associate with an analyte in a sample, thereby forming at least one captured analyte; b) a detection element including a target interacting probe associated with a first subunit of a detectable entity, wherein the target interacting probe can associate with the captured analyte so that a first complex is formed; and c) a second subunit that can complement the first subunit and generate a detectable entity, wherein the presence and/or amount of the analyte is indicated by detecting level or activity of the detectable entity.

### RELATED REFERENCES

This application claims priority to United States provisional patent application Ser. No. 61/780,713, filed Mar. 13, 2013, the entire contents of which are herein incorporated by reference.

### BACKGROUND

Development of robust methods that perform ultra-sensitive quantifications of analytes in diverse environments is a major challenge in analytical chemistry. Detection of single nucleic acids is of particular interest, and typically requests same form of signal amplification. Currently available signal amplification technologies such as, the enzyme-mediated detection methods typically used with immunoassays present poor assay confidence in quantifying the concentration of target nucleic acids. Sensitivity, dynamic range, and accuracy of such assays are mainly limited by nonspecific interactions of the reporter enzyme, thereby giving false positives, making such assays less sensitive than assays which amplify the genetic material (i.e., PCR based assays). There is a need for improved detection methodologies to quantify low levels of nucleic acids.

### SUMMARY

The present disclosure provides methodologies for detecting and measuring targets of interest (e.g., target nucleic acids) in samples. The provided technology is referred to as “Fragment Complementation” technology. Among other things, the present disclosure provides the insight that fragment complementation technology can beneficially be employed to bring the detection and measurement of analytes in samples down to a single molecule level. In some aspects, the present disclosure encompasses an assay utilizing a capture probe that interacts with a target of interest; a detection element comprising a target interacting probe associated with a first subunit of a detectable entity; and a second subunit that can complement the first subunit to generate a detectable entity.

The present disclosure further provides the insight that use of fragment complementation technology with encoding technologies permits simultaneous quantification of a plurality of different nucleic acids within a sample. In some embodiments, capture probes are immobilized on surfaces (e.g., particles) and individual surfaces are encoded. In some embodiments, individual surfaces are encoded optically.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only, not for limitation.

FIG. 1 illustrates principle of the Enzyme Fragment Complementation (EFC) assay in accordance with certain embodiments of the invention.

FIG. 2 illustrates exemplary steps in the EFC based assays in accordance with certain embodiments of the invention.

### DEFINITIONS

In order for the present invention to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “associated” as used herein, refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example, streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

The term “labeled” is used herein to describe a situation in which an entity (e.g., a nucleic acid probe, antibody, etc.) becomes detectable (e.g., visualizable), for example, by association with another entity (e.g., a nucleic acid, polypeptide, etc.) that comprises a detectable moiety. The detectable agent or moiety may be selected such that it generates a signal which can be measured. In some embodiments, a measurable feature (e.g., intensity) of the signal is related to the amount of a labeled entity. A wide variety of systems for labeling and/or detecting proteins and peptides are known in the art. Labeled proteins and peptides can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionucleotides, fluorophores, chemiluminescent agents, microparticles, enzymes (e.g., that catalyzes a reaction and generating one or more detectable entities), colorimetric labels, magnetic labels, haptens, molecular beacons, aptamer beacons, and the like.

The term “nucleic acid” as used herein, refers to a polymer of nucleotides. In some embodiments, nucleic acids are or contain deoxyribonucleic acids (DNA); in some embodiments, nucleic acids are or contain ribonucleic acids (RNA). In some embodiments, nucleic acids include naturally-occurring nucleotides (e.g., adenosine, thymidine, guanosine, cytidine, uridine, deoxyadenosine, deoxythymidine, deoxyguanosine, and deoxycytidine). Alternatively or additionally, in some embodiments, nucleic acids include non-naturally-occurring nucleotides including, but not limited to, nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups. In some embodiments, nucleic acids include phosphodiester backbone linkages; alternatively or additionally, in some embodiments, nucleic acids include one or more non-phosphodiester backbone linkages such as, for example, phosphorothioates and 5′-N-phosphoramidite linkages. In some embodiments, a nucleic acid is an oligonucleotide in that it is relatively short (e.g., less that about 5000, 4000, 3000, 2000, 1000, 900, 800, 700, 600, 500, 450, 400, 350, 300, 250, 200, 150, 100, 90, 80, 70, 60, 50, 45, 40, 35, 30, 25, 20, 15, 10 or fewer nucleotides in length).

The term “particles” as used herein, refers to discrete objects. Such objects can be of any shape or size. In some embodiments, some or all particles are substantially spherical. In some embodiments, utilized particles have sized within a defined range and/or showing a defined distribution. In some embodiments, particles having a diameter of less than 100 nanometers (nm) are also referred to as nanoparticles. Any of a variety of materials can be used to form or provide particles, as will be understood by those of skill in the art. In some embodiments, particular materials and/or shapes may be preferred based on chemistries or other features utilized in relevant embodiments; those of ordinary skill will be well familiar with various options and parameters guiding selection. In many embodiments, suitable materials include, but are not limited to, plastics, ceramics, glass, polystyrene, methylstyrene, acrylic polymers, metal, paramagnetic materials, thoria sol, graphitic carbon, titanium dioxide, latex or cross-linked dextrans such as Sepharose, cellulose, nylon, cross-linked micelles and teflon. In some embodiments, particles can be optically or magnetically detectable. In some embodiments, particles contain fluorescent or luminescent moieties, or other detectable moieties.

The term “polypeptide” as used herein, refers to a string of at least three amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, http://www.cco.caltech.edu/^{˜}dadgrp/Unnatstruct.gif, which displays structures of non-natural amino acids that have been successfully incorporated into functional ion channels) and/or amino acid analogs as are known in the art may alternatively be employed). For example, a polypeptide can be a protein. In some embodiments, one or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc.

The term “signal” used herein refers to a detectable and/or measurable event. In certain embodiments, a signal is detectable by the human eye, e.g., visible. In certain embodiments, detection of a signal requires an apparatus other than human eyes. In some embodiments, a signal may be or comprise electromagnetic radiation or a feature (e.g., wavelength, intensity). In some embodiments, a signal is an optical signal. A signal may be or comprises light (e.g., visible light and/or ultraviolet light). For example, a signal can be light generated by a chemiluminescent reaction. Typically, light can be detectable by a spectrophotometer. In some embodiments, a signal is or relates to radiation, e.g., radiation emitted by radioisotopes, infrared radiation, etc. In certain embodiments, a signal is a direct or indirect indicator of a property of a physical entity. For example, a signal could be used as an indicator of amount and/or concentration of a nucleic acid in a biological sample and/or in a reaction vessel.

The term “sample” as used herein refers to a volume or mass obtained, provided, and/or subjected to analysis. In some embodiments, a sample is or comprises a tissue sample, cell sample, a fluid sample, and the like. In some embodiments, a sample is taken from a subject (e.g., a human or animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid may be, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g., a subject); in some embodiments, a “sample” is the result of processing of a primary sample, for example to remove certain potentially contaminating components and/or to isolate or purify certain components of interest.

The term “substantially” as used herein refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” may be used herein to capture the potential lack of completeness inherent in many biological and chemical phenomena.

The term “subject” as used herein includes humans and mammals (e.g., mice, rats, pigs, cats, dogs, and horses). In many embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be , for example, rodents (e.g., mice, rats, hamsters), rabbits, primates, or swine such as inbred pigs and the like.

The term “target nucleic acid” as used herein, refers to one or more nucleic acid molecules to be detected and/or quantified in accordance with the present invention. Exemplary target nucleic acids include, but not limited to DNA, RNA, miRNA, and cDNAs. In some embodiments, a target nucleic acid comprises a plurality of different nucleic acid molecules (i.e., having different nucleotide sequences); in some embodiments, only a single nucleic acid molecule is a target. In some embodiments, target nucleic acids are of the same origin (e.g., from the same chromosome, genomic locus, or gene, although the molecules may come from one individual, or multiple individuals, or more than one type of cells, such as tumor cells, placental cells, blood cells, etc.).

### DETAILED DESCRIPTION OF CERTAIN EMBODIMENTS

The present disclosure provides, among other things, methods and compositions for detecting and/or quantifying analytes (e.g., target nucleic acids) using fragment complementation technologies. Enzyme fragment complementation (EFC) is particularly useful in some embodiments of the present invention. It is contemplated that the methods described herein can be performed in a number of different formats using a variety of different detectable labels, reagents, reaction conditions, and detection systems.

The present disclosure provides a method for detecting the presence and/or abundance of analyte(s) in a sample by a) contacting a sample comprising at least one analyte with at least one capture probe under conditions and for a time sufficient for the analyte to associate with the capture probe, thereby forming at least one captured analyte; b) contacting the at least one captured analyte with at least one detection element, comprising a target interacting probe associated with a first subunit of a detectable entity, capturing being performed under conditions and for a time sufficient for the captured analyte to associate with the target interaction probe, so that at least one first complex, comprising the capture probe, the analyte, the target interacting probe, and the first subunit is formed; c) contacting the at least one first complex with at least one second subunit that, when associated with the first subunit, complements the first subunit and generates the detectable entity, under conditions and for a time sufficient for the first and second subunit to associate and generate the detectable entity; and d) determining presence and/or amount of the analyte indicated by detecting level or activity of the detectable entity directly or indirectly.

In some embodiments, one or more detectable entities are used in accordance with the present disclosure; each independently having an enzyme activity, and/or each being independently labeled to facilitate direct and/or indirect detection.

In some embodiments, one or more capture probes suitable for the inventive methods and compositions are attached to a surface. Exemplary surfaces includes a microarray and/or a particle. In addition, it is contemplated that such fragment complementation technologies based approach may be used with encoded surfaces.

As described herein, one feature of the present invention is the recognition that fragment complementation technologies can be usefully utilized in nucleic acid application (e.g., assays).

Various aspects of the invention are described in further detail in the following subsections. The use of subsections is not meant to limit the invention. Each subsection may apply to any aspect of the invention.

### Fragment Complementation and Assays

Fragment complementation, as described herein, refers to the assemblage of two or more subunits to create a whole. In accordance with the present invention, subunits of a detectable entity (e.g., any agent that, directly or indirectly generates or associates with a signal) are provided in a separated state (i.e., separated from one another), so that no signal or only background signal of inactive subunits is present. The subunits are brought together through association of subunits with one another to generate a detectable entity (e.g., an active form of enzyme), and also direct or indirect association of at least one subunit with an analyte, so that presence of the analyte ultimately leads to generation of a signal. In some embodiments, a signal itself, or some feature of the signal (e.g., identity, level, intensity, frequency, wavelength, etc) correlates with presence or amount of the analyte. In some embodiments, quantification of the signal or one or more features thereof achieves or permits quantification of the analyte.

### Detectable Entities

As will be appreciated by those skilled in the art, detectable entities comprised of separable subunits may be of any chemical class (e.g., proteins, nucleic acids, carbohydrates, lipids, small molecules, vitamins, minerals, or combinations thereof). Detectable entities formed by fragment complementation as described herein are “detectable” in that their presence or level results, directly or indirectly, in the production of a signal.

In some embodiments, a detectable entity for use in accordance with the present invention comprises one or more polypeptide subunits; in some such embodiments fragment complementation comprises association of such polypeptide subunits with one another to form the detectable entity.

In some embodiments, a detectable entity for use in accordance with the present invention comprises an enzyme; in some such embodiments, fragment complementation comprises association of inactive enzyme subunits with one another to form the active enzyme. In some embodiments, an active enzyme formed by association of subunits as described herein has at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100% or more of the activity of a reference enzyme. In some embodiments, a reference enzyme is a native enzyme (e.g., a naturally occurring enzyme). In some such embodiments, a reference enzyme is a native enzyme that naturally contains the subunits; in some such embodiments the reference enzyme is a native enzyme that naturally contains the subunits as part of a single molecular entity (i.e., in covalent association with one another).

In some embodiments, each subunit of a detectable entity as described herein comprises approximately 50% of the detectable entity. In some embodiments, one subunit comprises a larger portion of the detectable than the other. For example, in some embodiments, one subunit of a detectable entity may comprise approximately 90%, approximately 85%, approximately 80%, approximately 75%, approximately 70%, approximately 65%, approximately 60%, or approximately 65% of the detectable entity.

In some embodiments, a detectable entity is formed by association of more than two subunits, each of which may represent an approximately equal percentage (e.g., 33%, 25%, 20%, etc., depending on the number of subunits) of the whole, or may represent a larger or smaller percentage of the whole as compared with the other subunits.

In some embodiments, association of detectable entity subunits requires and/or is benefits from participation of one or more other agents. To give but one example, accessory proteins (including but not limited to chaperone-type proteins) participate in and/or stabilize complexes between or among polypeptides. Those of skill in the art would be aware of other contexts where accessory proteins, or other agents may be useful in helping detectable entity associations as described herein.

In some embodiments, particular detectable entities that can be formed by fragment complementation of subunits as described herein are detectable proteins such as Prostate-specific antigen (PSA), troponin, HIV protease, etc. Detectable proteins can be detected directly or indirectly. In some embodiments, particular detectable entities that can be formed by fragment complementation of subunits as describe herein are detectable enzymes. Exemplary detectable enzymes include, but are not limited to, β-galactosidase, dihydrofolate reductase (“DHFR”), horse radish peroxidase, β-lactamase, luciferase, etc.

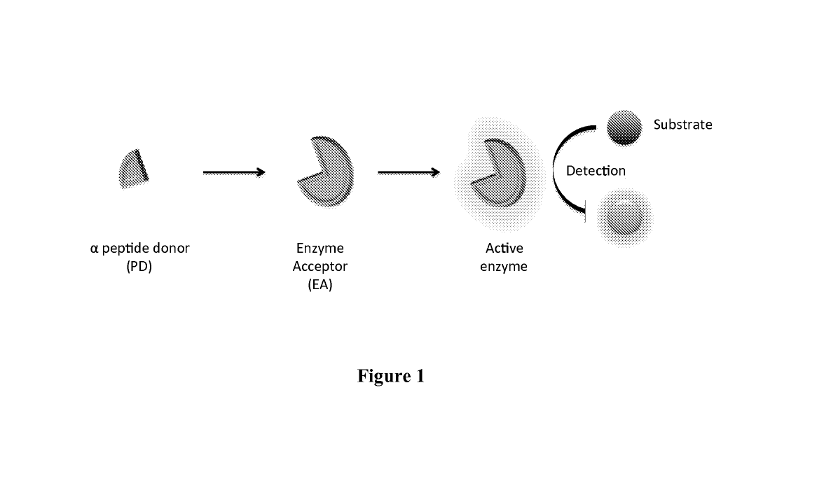

In some particular embodiments, the present invention utilizes enzyme fragment complementation (EFC) of β-galactosidase. β-galactosidase can be separated into amino-(“acceptor”) and carboxyl-(“donor”) terminal fragments that are each inactive but that complement each other to restore enzymatic activity when combined so that they associate with one another (see FIG. 1).

### Target Nucleic Acids

According to some embodiments of the present disclosure, analytes can be any atom, molecule, ion, molecular ion, compound, particle, cell, or virus to be either detected or evaluated. Exemplary analytes can include, but are not limited to, an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including nucleic acids, enzymes, hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc.

In some embodiments, analytes are nucleic acids. Target nucleic acids may be any form of DNA, RNA, or any combination thereof. In certain embodiments of the present invention, a target nucleic acid may be or contain a portion of a gene, a regulatory sequence, genomic DNA, cDNA, RNA including mRNA and rRNA, or any combination thereof. In some embodiments, a target nucleic acid may be or contain a single or double stranded RNA or DNA, including, for example, gDNA, cDNA, mRNA, pre-mRNA, miRNA, etc. Furthermore, in some embodiments, a target nucleic acid may include one or more residues that is an analog of a naturally-occurring nucleotide. In some embodiments, such analogs have a backbone other than a phosphodiester backbone. For example, the so-called “peptide nucleic acids,” which are known in the art and have peptide bonds instead of phosphodiester bonds in the backbone, may be considered to be “target nucleic acids” in accordance with certain embodiments of the invention.

Target nucleic acids can be naturally or synthetically produced, including produced using recombinant expression systems, chemically synthesized, etc. Where appropriate, e.g., in the case of chemically synthesized molecules, nucleic acids can comprise nucleoside analogs.

In some embodiments, a target nucleic acid has a nucleotide sequence known in advance. In some embodiments, a target nucleic acid has a nucleotide sequence known to be present in a microorganism (e.g., bacterium, yeast, fungus, etc), virus, or other infectious agent or parasite. In some embodiments, a target nucleic acid has a nucleotide sequence of E coli H70157 (food poisoning), HPV, HIV, etc.

In some embodiments of the present invention, target nucleic acids are microRNAs such as, for example, certain microRNAs demonstrated herein to be indicative of certain diseases, disorders, or conditions, including for example, cancer, diabetes, Alzheimer's, cardiovascular disease, etc. Certain exemplary potential target microRNAs include, for example let-7a, miR-21, miR-29b-2, miR-181b-1, miR-143, miR-145, miR-146a, miR-210, miR-221, miR-222, miR-10b, miR-15a, miR-16, miR-17, miR-18a, miR-19a, miR20a, miR-1, miR-29, miR-181, miR372, miR-373, miR-155, miR-101, miR-195, miR-29, miR-17-3p, miR-92a, miR-25, miR-223, miR-486, miR-223, mir-375, miR-99b, miR-127, miR-126, miR-184.

In some embodiments, one or more target nucleic acid are obtained from a sample. A sample can be obtained or prepared from any appropriate source. In some embodiments, for example, a sample is or comprises a tissue sample. In some embodiments, a sample is or comprises an environmental sample. In some embodiments, a sample is or comprises a chemical reaction (e.g., a chemical synthesis reaction).

In some embodiments, a sample is subjected to one or more isolation steps, e.g., to separate target nucleic acids from one or more other components present in the sample. In some embodiments, an isolation step separates target nucleic acids from non-nucleic acid components of a sample. In some embodiments, an isolation step separates target nucleic acids from one or more other nucleic acids, whether on the basis of chemical identity (e.g., DNA vs RNA) and/or sequence.

In some embodiments, a sample is subjected to one or more modification steps. In some embodiments, target (and/or non-target) nucleic acids in a sample are modified, for example, associated with an entity, prior to, during or even after detection. For example, a target can be labeled. In some embodiments, a sample is modified to generate nucleic acids that can be detected. For example, a sample containing cells can be modified by cell lysis and/or DNA extraction.

In some embodiments, a sample is subjected to one or more amplification steps. For example, target nucleic acids from a sample can be amplified via PCR, prior to or during detection.

### Probes

A probe can be any molecule, compound, or solid support modification that can be used to associate (e.g., probe for, attach or bind) an analyte. In some embodiments, a probe can be capture probe and/or a target interacting probe as used in the present disclosure.

As will be appreciated by those in the art, the composition of probes will depend on the composition of analytes. Probes for a wide variety of analytes are known or can be readily found using known techniques. In some embodiments, when an analyte is a protein, probes can include proteins (particularly including antibodies or fragments thereof (FAbs, etc.)) or small molecules. Exemplary protein probes include peptides. In some embodiments, when an analyte is an enzyme, probes include substrates and inhibitors. Suitable analyte/probe pairs include, but are not limited to, antibodies/antigens, receptors/ligands, proteins/nucleic acid, enzymes/substrates and/or inhibitors, carbohydrates (including glycoproteins and glycolipids)/lectins, proteins/proteins, proteins/small molecules; and carbohydrates and their binding partners are also suitable analyte-probe pairs.

Probes in accordance with some embodiments of the present disclosure are polynucleotide molecules, and typically oligonucleotide molecules whose sequence permits hybridization with sites within target nucleic acids.

In some embodiments, a probe has a length greater than 10 bases, 20 bases, 30 bases, 50 bases, 80 bases, 100 bases, 150 bases, 200 bases, 300 bases, 400 bases, 500 bases, 600 bases, 700 bases, 800 bases, 900 bases or 1000 bases. In some embodiments, a probe has a length less than 5 bases, 10 bases, 20 bases, 30 bases, 50 bases, 80 bases, 100 bases, 150 bases, 200 bases, 300 bases, 400 bases, 500 bases, 600 bases, 700 bases, 800 bases, 900 bases or 1000 bases. In some embodiments, a probe has a length within a range of 5-1000 bases, 10-500 bases, or 100-200 bases. In some embodiments, a probe has a length within a range of any two values above.

In some embodiments, a probe has a nucleotide sequence that shows at least 50%, 55%, 60%, 65%, 70%, 75%, 80%, 85%, 90%, 91%, 92%, 93%, 94%, 95%, 96%, 97%, 98%, 99%, or even 100% identical to a complement of a particular known target nucleic acid sequence (i.e., to a site within a target nucleic acid). In some embodiments, a probe hybridizes specifically with the target site under even under stringent hybridization conditions.

In some embodiments, probe has a nucleotide sequence that distinguishes between different possible target nucleic acids, including those that show very high sequence identity with one another.

### Surfaces

In some embodiments, a capture probe can be immobilized on a surface. Such a surface suitable for use in accordance with the present disclosure can be or comprise, for example, a particle, bead, planar surface and the like.

Where particles are used in the practice of the present invention, it is not intended that the present invention be limited to a particular type. A variety of particle types are commercially available, including but not limited to, particles selected from agarose beads, streptavidin-coated beads, NeutrAvidin-coated beads, antibody-coated beads, paramagnetic beads, magnetic beads, electrostatic beads, electrically conducting beads, fluorescently labeled beads, colloidal beads, glass beads, semiconductor beads, and polymeric beads.

Particles used in accordance with some embodiments of the present invention need not be spherical; irregular particles and/or particles having non-spherical shapes, may be used. Particles can have a variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (particles having four leg-like appendages), triangles, prisms, etc.

A particle is typically an entity having a greatest dimension (e.g. diameter) of less than 1000 microns (um). In some embodiments, particles have a greatest dimension of less than 500 μm, 200 μm, 100 μm, 50 μm, 10 μm, 5 μm or 1 μm. In some embodiments, particles have a greatest dimension of less than 900 nm, 800 nm, 700 nm, 600 nm, 500 nm, 400 nm, 300 nm, 200 nm, or 100 nm. Smaller particles, e.g., having a greatest dimension of 50 nm or less are used in some embodiments of the invention. In some embodiments, particles have a greatest dimension ranging between 1 μm and 10 μm. In some embodiments, particles have a greatest dimension ranging between any two values above. A population of particles can be but need not be relatively uniform in terms of size, shape, and/or composition.

In some embodiments, polymeric particles may be used in accordance with the present invention. For example, particles can be made of organic polymer including, but not limiting to, polystyrene, polymethylmethacrylate, polyacrylamide, poly(vinyl chloride), carboxylated poly(vinyl chloride), poly(vinyl chloride-co-vinyl acetate-co-vinyl alcohol), and combination thereof. Additionally or alternatively, particles can be or comprises inorganic polymers such as silica (SiO₂).

In some embodiments, particles can be labeled. In some embodiments, particles are functionalized (e.g., surface functionalized by adsorption or covalently bonding) or “doped” or “loaded” with fluorescent and luminescent moieties (e.g., fluorescent dyes) for optical encoding of particles. Examples of fluorescent dyes include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. In addition to or alternative to single optical moieties, encoding can be accomplished in a ratio of at least two moieties. In certain embodiments, optically detectable particles comprise a hologram.

In addition or alternatively, particles are or comprise intrinsically fluorescent or luminescent particles. In certain embodiments, particles are or comprise quantum dots (QDs). QDs are bright, fluorescent nanocrystals with physical dimensions small enough such that the effect of quantum confinement gives rise to unique optical and electronic properties. Semiconductor QDs are often composed of atoms from groups II-VI or III-V in the periodic table, but other compositions are possible. By varying their size and composition, the emission wavelength can be tuned (i.e., adjusted in a predictable and controllable manner) from the blue to the near infrared. In certain embodiments, detectable particles are or comprise metal particles. Metals of use include, but are not limited to, gold, silver, iron, cobalt, zinc, cadmium, nickel, gadolinium, chromium, copper, manganese, palladium, tin, and alloys thereof Oxides of any of these metals can be used.

Certain metal particles, referred to as plasmon resonant particles, exhibit the well known phenomenon of plasmon resonance. The features of the spectrum of a plasmon resonant particle (e.g., peak wavelength) depend on a number of factors, including the particle's material composition, the shape and size of the particle, the refractive index or dielectric properties of the surrounding medium, and the presence of other particles in the vicinity. Selection of particular particle shapes, sizes, and compositions makes it possible to produce particles with a wide range of distinguishable optically detectable properties thus allowing for concurrent detection of multiple nucleic acids by using particles with different properties such as peak scattering wavelength.

Magnetic properties of particles can be used in accordance with the present invention. Particles in some embodiments are or comprise magnetic particles, that is, magnetically responsive particles that contain one or more metals or oxides or hydroxides thereof Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the aforementioned oxides or hydroxides, and mixtures of any of the foregoing.

### Detection Methods and Kits

Any appropriate means and/or system can be utilized in accordance with the present disclosure to detect and/or quantify captured analytes (e.g., target nucleic acids) based on a detectable entity generated by fragment complementation. Those of ordinary skill in the art will appreciate that, in some embodiments, actual detection or development of a generated or changed detectable property of a detectable entity may require or involve one or more additional steps (e.g., association of a labeled moiety with detectable entity).

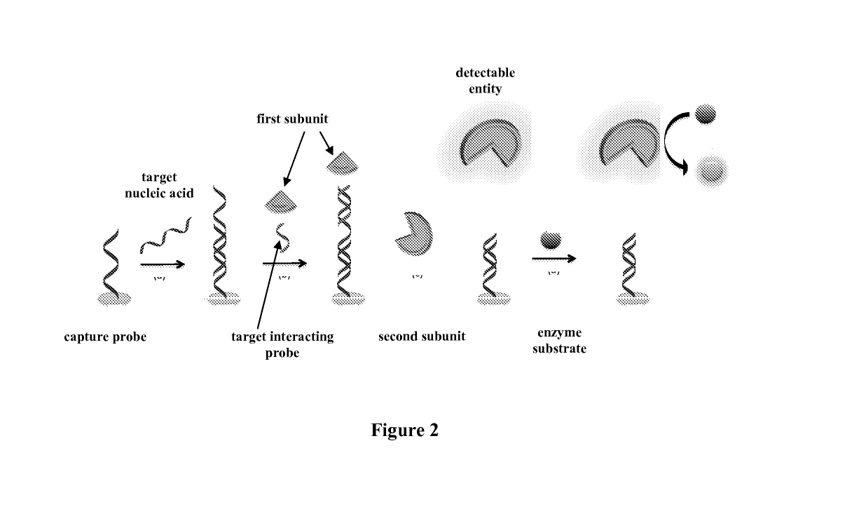

According to some embodiments in which a detectable entity is an enzyme, a chromogenic, fluorogenic, or chemiluminescent enzyme substrate is contacted with the enzyme to produce a detectable product. Any known chromogenic, fluorogenic, or chemiluminescent enzyme substrate capable of producing a detectable product in a reaction with a particular enzyme can be used in the present invention, including any of the chromogenic, fluorogenic, or chemiluminescent enzyme substrates disclosed in The Handbook—A Guide to Fluorescent Probes and Labeling Technologies, Tenth Ed, Chapter 10, http://probes.invitrogen.com/handbook/sections/1000.html, which is incorporated herein by reference in its entirety. Referring to FIG. 2, an analyte can be detected using a sandwich assay as described further herein in Example 1, which the enzyme is β-galactosidase, an enzyme substrate added can be a β-galactosidase substrate such as resorufin β-D-galactopyranoside.

In some embodiments, a detectable property (aspect) is optical. Exemplary optical properties include, but are not limited to, fluorescent, ultraviolet, infrared, holographic, radiographic signals and any combination thereof. An optical property, in some embodiments, can be detected through absorption, emission, reflection, refraction, interference, diffraction, dispersion, scattering, or any combination thereof, etc. In addition or alternatively, electrochemical detection can be used in accordance with some embodiments of the present invention, in which an electrochemically detectable product (e.g. H₂O₂) is generated.

In some embodiments, detection and/or quantification can comprise a step of counting the number of surfaces such as particles, onto which an analyte is captured. Such counting can determine the quantity of teh analyte in samples. In some embodiments, a population of particles as described above used in accordance with the present invention has more than one subgroup of particles. A subgroup of particles can share a signature on individual particles in the subgroup to be differentiated from another subgroup of particles. Such encoding enables multiplexed analysis of more than one type of analytes.

It is not intended that the present disclosure be limited to a particular coding scheme. A signature for encoding can be a visually detectable feature such as, for example, color, apparent size, or visibility (i.e. simply whether or not the particle is “visible”, or optically detectable, under particular conditions). Such visibility, as will be understood by those skilled in the art, can include, for example, presence or amount of electromagnetic radiation at one or more particular frequencies, presence or identity of a particular holographic signature, presence or amount of radioactivity, etc. In various embodiments of the present invention, an optical signature of a particle is used for encoding. Detailed description of optically interrogatable encoding can be found, for example, in United States patents U.S. Pat. No. 6,023,540 and U.S. Pat. No. 6,327,410, the contents of which are incorporated herein by reference.

A variety of assays known in the art can be used in accordance with the present disclosure. Also provided are kits for carrying out the methods and/or assays described herein.

In some embodiments, a kit comprises reagents or other materials for preparing samples and/or performing methods, including, for example, reporting dyes, probes, detergents, solvents, or ion exchange resins. In some embodiments, a kit comprises one or more reagents for optical detection.

A kit may include instructions pertinent for the particular embodiment of the kit, such instructions describing incubation and/or amplification conditions for operation of assays. A kit may comprise reaction containers such as microcentrifuge tubes, microtiter plates, and the like.

In some embodiments, a kit further comprises instructions for analysis, interpretation and/or dissemination of data acquired by the kit. In some embodiments, instructions for the operation, analysis, interpretation and dissemination of data of a kit are provided on computer readable media.

### Applications

The present invention has many applications, including, but not limited to, diagnosis and monitoring in medicine and any non-medical applications, where the presence and/or the amount of a target can be determined In some embodiments, the presence or the amount of a target nucleic acid is determined using the present invention.

Those of ordinary skill reading the present disclosure, will appreciate its broad applicability. In some embodiments, provided methods herein are used to detect and/or quantify target nucleic acids, for example, to profile a specific tissue or a specific condition. In some embodiments, provided methods herein are used to detect and/or quantify target nucleic acids to detect biomarkers for specific tissue or condition. In certain embodiments, provided methods herein are used to detect and/or quantify target nucleic acids to profile a neoplastic and/or cancer cell.

For example, a wide variety of infectious diseases can be detected and/or determined by the process of the present invention, for example, those caused by bacterial, viral, parasite, and fungal infectious agents. The resistance of various infectious agents to drugs can also be determined using the present invention.

Representative bacterial infectious agents which can be detected and/or determined by the present invention include, but are not limited to, Escherichia coli, Salmonella, Shigella, Klebsiella, Pseudomonas, Listeria monocytogenes, Mycobacterium tuberculosis, Mycobacterium aviumintracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes.

Representative fungal infectious agents which can be detected and/or determined by the present invention include, but are not limited to, Cryptococcus neoformans, Blastomyces dermatitidis, Histoplasma capsulatum, Coccidioides immitis, Paracoccidioides brasiliensis, Candida albicans, Aspergillus fumigautus, Phycomycetes (Rhizopus), Sporothrix schenckii, Chromomycosis, and Maduromycosis.

Representative viral infectious agents which can be detected and/or determined by the present invention include, but are not limited to, human immunodeficiency virus, human T-cell lymphocytotrophic virus, hepatitis viruses (e.g., Hepatitis B Virus and Hepatitis C Virus), Epstein-Barr Virus, cytomegalovirus, influenza viruses, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

Representative parasitic agents which can be detected and/or determined by the present invention include, but are not limited to, Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

The present invention can also be useful for detection and/or determination of drug resistance by infectious agents. For example, vancomycin-resistant Enterococcus faecium, methicillin-resistant Staphylococcus aureus, penicillin-resistant Streptococcus pneumoniae, multi-drug resistant Mycobacterium tuberculosis, and AZT-resistant human immunodeficiency virus can be identified with the present invention.

Genetic diseases can also be detected and/or determined by the process of the present invention. This can be carried out by prenatal or post-natal screening for chromosomal and genetic aberrations or for genetic diseases. Examples of detectable genetic diseases include, but are not limited to: 21 hydroxylase deficiency, cystic fibrosis, Fragile X Syndrome, Turner Syndrome, Duchenne Muscular Dystrophy, Down Syndrome or other trisomies, heart disease, single gene diseases, HLA typing, phenylketonuria, sickle cell anemia, Tay-Sachs Disease, thalassemia, Klinefelter Syndrome, Huntington Disease, autoimmune diseases, lipidosis, obesity defects, hemophilia, inborn errors of metabolism, and diabetes.

Cancers which can be detected and/or determined by the process of the present invention generally involve oncogenes, tumor suppressor genes, or genes involved in DNA amplification, replication, recombination, or repair. Examples of these include, but are not limited to: BRCA1 gene, p53 gene, APC gene, Her2/Neu amplification, Bcr/Ab1, K-ras gene, and human papillomavirus Types 16 and 18. Various aspects of the present invention can be used to identify amplifications, large deletions as well as point mutations and small deletions/insertions of the above genes in the following common human cancers: leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, head and neck tumors, and cervical neoplasms.

In the area of environmental monitoring, the present invention can be used, for example, for detection, identification, and monitoring of pathogenic and indigenous microorganisms in natural and engineered ecosystems and microcosms such as in municipal waste water purification systems and water reservoirs or in polluted areas undergoing bioremediation and/or beaches. It is also possible to detect plasmids containing genes that can metabolize xenobiotics, to monitor specific target microorganisms in population dynamic studies, or either to detect, identify, or monitor genetically modified microorganisms in the environment and in industrial plants.

The present invention can be used in a variety of forensic areas, including, for example, for human identification for military personnel and criminal investigation, paternity testing and family relation analysis, HLA compatibility typing, and screening blood, sperm, or transplantation organs for contamination.

In some embodiments, the present invention is useful for detection and/or determination of bioterrorism agents/diseases. For example, Anthrax, Botulism, Plague, Smallpox, Tularemia and Viral hemorrhagic fevers can be identified with the present invention. In addition or alternatively, Brucellosis, Epsilon toxin of Clostridium perfringens, Glanders, Melioidosis, Psittacosis, Q fever, Ricin toxin from Ricinus communis, Staphylococcal enterotoxin B, Typhus fever, viral encephalitis (alphaviruses [e.g., Venezuelan equine encephalitis, eastern equine encephalitis, western equine encephalitis]), water safety threats (e.g., Vibrio cholerae, Cryptosporidium parvum), and emerging infectious diseases such as Nipah virus and hantavirus can be identified with the present invention. More information of bioterrorism agents/diseases can be found on http://www.bt.cdc.gov/agent/agentlist-category.asp.

In the food and feed industry, the present invention has a wide variety of applications. For example, it can be used for identification and characterization of production organisms such as yeast for production of beer, wine, cheese, yoghurt, bread, etc. Another area of use is with regard to quality control and certification of products and processes (e.g., livestock, pasteurization, and meat processing) for contaminants. Other uses include the characterization of plants, bulbs, and seeds for breeding purposes, identification of the presence of plant-specific pathogens, and detection and identification of veterinary infections.

### EXEMPLIFICATION

### Example 1

According to the present invention, provided fragment complementation based methodologies are particularly useful in quantifying nucleic acids. In this Example, EFC technology was utilized for detection of nucleic acid molecules in a sandwich-type assay. This assay generates a detectable entity (e.g., an enzyme), thus involving no addition of the detectable entity. Such a detectable entity can be generated when a target analyte is bound to a capture probe and a secondary probe associate with a donor that is coupled with an acceptor via fragment complementation (e.g., EFC). In various embodiments, it is beneficial to utilize the provided method and composition herein because the complication of non-specific binding of protein moieties, as seen in conventional enzymatic assays, can be substantially eliminated.

As depicted in FIG. 2, a fragment complementation based assay can be performed as follows. A fluid sample is exposed to capture probes immobilized on a surface to capture target nucleic acid(s) in the sample. The captured target nucleic acid(s) are hybridized to an α-fragment peptide-labeled secondary probe (donor) and the resulting complexes are exposed to inactive mutant β-galactosidase (acceptor) resulting in an active enzyme via EFC. The detection of the presence of the analyte (e.g., target nucleic acids) in the fluid sample can be accomplished by tracking the luminescence signal of the active enzyme in contact with an enzyme substrate.

### Other Embodiments and Equivalents

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A method comprising:
a) contacting a sample comprising at least one analyte with at least one capture probe immobilized on a surface under conditions and for a time sufficient for the analyte to associate with the capture probe, thereby forming at least one captured analyte;
b) contacting the at least one captured analyte with at least one detection element, comprising a target interacting probe associated with a first subunit of a detectable entity, capturing being performed under conditions and for a time sufficient for the captured analyte to associate with the target interaction probe, so that at least one first complex, comprising the capture probe, the analyte, the target interacting probe, and the first subunit is formed;
c) contacting the at least one first complex with at least one second subunit that, when associated with the first subunit, complements the first subunit and generates the detectable entity, under conditions and for a time sufficient for the first and second subunit to associated and generate the detectable entity; and
d) determining presence and/or amount of the analyte indicated by detecting level or activity of the detectable entity.

2. The method of claim 1, wherein the analyte is a target nucleic acid.

3. The method of claim 2, wherein the target nucleic acid is selected from the group consisting of DNA, RNA, miRNA, cDNA and any combination thereof.

4. The method of claim 3, wherein the target nucleic acid is miRNA.

5. The method of claim 3, wherein the detectable entity is an enzyme.

6. The method of any one of claims 1-5, wherein the enzyme is selected from the group consisting of β-galactosidase, dihydrofolate reductase (“DHFR”), horse radish peroxidase, β-lactamase and luciferase.

7. The method of any one of claims 5 and 6, wherein the step d) comprises contacting with an enzyme substrate.

8. The method of claim 7, wherein the enzyme is β-galactosidase and the enzyme substrate is resorufin β-D-galactopyranoside.

9. The method of any one of claims 1-9, wherein the surface is a particle.

10. The method of claim 9, wherein the particle is encoded.

11. The method of claim 10, further comprising a step of decoding the encoded particle.

12. The method of any one of claims 1-11, wherein the step d) is performed by flow cytometry.

13. The method of any one of claims 1-11, wherein the step d) is performed by imaging.

14. The method of any one of claims 1-13, wherein the sample is selected from the group consisting of blood, plasma, serum, saliva, tissue and any combination thereof.

15. The method of any one of claims 1-13, wherein the sample is from a cancer patient.

16. The method of any one of claims 2-13, wherein the target nucleic acid is or comprises at least a portion of a gene related to a genetic disease or a genetic polymorphism.

17. The method of any one of claims 2-13, wherein the target nucleic acid is or comprises at least a portion of an oncogene or a tumor suppressor gene.

18. The method of any one of claims 2-13, wherein the target nucleic acid is or comprises at least a portion of a virus genome.

19. A method comprising:
a) contacting a sample comprising one or more target nucleic acids with one or more capture oligonucleotide probes immobilized on a surface under conditions that permit the target nucleic acids to hybridize with the capture oligonucleotide probes, thereby forming one or more captured target nucleic acids;
b) contacting one or more detection elements each comprising a target interacting probe associated with an a-fragment peptide, so that the detection elements interact with the captured target nucleic acids under conditions that permit the captured target nucleic acids to hybridize with the target interaction probes, thereby forming one or more oligocomplexes;
c) contacting the oligocomplexes with one or more inactive mutant β-galactosidases under conditions that permit complementation of the α-fragment peptides of the oligocomplexes with the inactive mutant β-galactosidases, thereby generating one or more enzymatically active entities each comprising an activated β-galactosidase unit; and
d) determining presence and/or amount of the target nucleic acids by detecting enzyme activity of the activated β-galactosidase unit.

20. The method of any one of claims 1-19, wherein the method is used to detect a infectious disease.

21. The method of claim 20, wherein the infectious disease is caused by a bacterial infectious agent selected from the group consisting of Escherichia coli, Salmonella, Shigella, Klebsiella, Pseudomonas, Listeria monocytogenes, Mycobacterium tuberculosis, Mycobacterium aviumintracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes.

22. A kit comprising:
a) one or more copies of at least one capture probe immobilized on a surface, wherein the capture probe is characterized by an ability to associate with an analyte in a sample under predetermined conditions, thereby forming at least one captured analyte;
b) at least one detection element, comprising a target interacting probe associated with a first subunit of a detectable entity, wherein the target interacting probe is characterized by an ability to associate with the captured analyte under predetermined conditions so that at least one first complex, comprising the capture probe, the analyte, the target interacting probe, and the first subunit is formed; and
c) at least one second subunit being characterized by an ability to, when associated with the first subunit, complements the first subunit and generates a detectable entity, under predetermined conditions, wherein the presence and/or amount of the analyte is indicated by detecting level or activity of the detectable entity.

23. A kit comprising:
a) a population of encoded surfaces comprising one or more sub-populations, wherein the sub-populations differing from one another in that:
i) each sub-population has a signature distinguishable from one another; and
ii) each sub-population carriers one or more copies of a single capture probe, wherein the capture probe is characterized by an ability to associate with a particular one of one or more analytes in a sample under predetermined conditions, thereby forming a particular captured analyte;
b) one or more detection elements, each comprising a target interacting probe associated with a first subunit of a detectable entity, wherein the target interacting probe is characterized by an ability to associate with the particular captured analyte under predetermined conditions so that the particular first complex, comprising the capture probe, the particular analyte, the target interacting probe, and the particular first subunit is formed; and
c) one or more second subunits each being characterized by an ability to, when associated with the first subunit, complements the first subunit and generates a particular detectable entity, under predetermined conditions, wherein the presence and/or amount of the particular analyte is indicated by detecting level or activity of the particular detectable entity.

24. The kit of claim 22 or 23, further comprising at least one additional reagents.

25. The kit of claim 24, wherein the detectable entity is an enzyme and the additional reagents comprise an enzyme substrate.
